(19) United States
(12) Patent Application Publication
(10) Pub. No.: US 2007/0021773 A1
(43) Pub. Date: Jan. 25, 2007

(54) Nasal-dilating device for a constricted nostril

(76) Inventor: Paul H. Nolte, Richmond, VA (US)

Correspondence Address: DONALD R. SCHOONOVER, 4211 ROLLING HILLS DRIVE, NIXA, MO, 65714-8771, US

(21) Appl. No.: 11/184,146

(22) Filed: Jul. 20, 2005

(51) Int. Cl. A61M 29/00

(52) U.S. Cl. 606/199

(57) ABSTRACT

An inconspicuous nasal-dilating device for a constricted nostril, the device being insertable into and frictionally retained in the constricted nostril. The device has a circular cross-sectional shape that is laterally squeezable to an oval shape for purposes of inserting the device into the constricted nostril and, upon being released from the oval shape, attempts to return to its circular cross-sectional shape thereby dilating an air passageway of the constricted nostril.

### BACKGROUND OF THE INVENTION

1. Field of the Invention

The present invention relates to medical devices and, more specifically without limitation, to breathing devices.

2. Description of the Related Art

Many people have difficulty breathing through one or both nostrils because the air passageway therethrough is constricted, such as due to an earlier injury or a deviated septum for example. Although various prior art devices have been developed to open up a constricted passageway to promote easier breathing, most if not all of such prior art devices are very conspicuous causing enough embarrassment and anxiety to users thereof such that the use of such devices are largely avoided.

What is needed is an inconspicuous nasal-dilating device for a constricted nostril.

### SUMMARY OF THE INVENTION

The improvements of the nasal-dilating device for a constricted nostril of the present invention include a device comprising a body unit having a sidewall, an open inner end and an open outer end such that air can freely pass through the device, the body unit having unitary construction and being formed of flexible and resilient material. The body unit includes a first portion having a circular cross-sectional configuration, and a second portion spaced in end to end relation with the first portion, the second portion also having a circular cross-sectional configuration and extending radially outwardly from the first portion. The body unit is dimensioned to be insertable into, and frictionally retained in, a constricted nostril wherein the body unit is laterally squeezable to an oval shape for purposes of inserting the body unit into the constricted nostril and, upon being released from the oval shape, attempts to return to the circular cross-sectional shape thereof, thereby opening the air passageway through the constricted nostril.

The improvements of a modified embodiment of the nasal-dilating device of the present invention include the body unit having an inner end and an outer end, and the body unit flares continuously outwardly from the inner end to the outer end.

### PRINCIPAL OBJECTS AND ADVANTAGES OF THE INVENTION

The principal objects and advantages of the present invention include: providing an inconspicuous nasal-dilating device for a constricted nostril; providing such a device that is insertable into and frictionally retained in a constricted nostril; providing such a device that has a circular cross-sectional shape and that is laterally squeezable to an oval shape for purposes of inserting the device into a constricted nostril and, upon being released from the oval shape, attempts to return to its circular cross-sectional shape; and generally providing such a device that is reliable in performance, capable of long lasting life, and particularly well adapted for the proposed usages thereof.

Other objects and advantages of this invention will become apparent from the following description taken in conjunction with the accompanying drawings wherein are set forth, by way of illustration and example, certain embodiments of this invention.

### BRIEF DESCRIPTION OF THE DRAWING FIGURES

FIG. 1 is perspective view of a nasal-dilating device for a constricted nostril in accordance with the present invention.

FIG. 2 is a cross-sectional view of the nasal-dilating device for a constricted nostril, taken along line 2-2 of FIG. 1.

FIG. 3 is perspective view of a modified embodiment of the nasal-dilating device for a constricted nostril in accordance with the present invention.

### DETAILED DESCRIPTION OF THE INVENTION

As required, embodiments of the present invention are disclosed herein, however, it is to be understood that the disclosed embodiments are merely exemplary of the invention, which may be embodied in various forms. Therefore, specific structural and functional details disclosed herein are not to be interpreted as limiting, but merely as a basis for claims and as a representative basis for teaching one skilled in the art to variously employ the present invention in virtually any appropriately detailed structure.

The device of the present invention is designed to be inserted into a constricted nostril of a user and to dilate that constricted nostril so the user can breathe more easily through the constricted nostril.

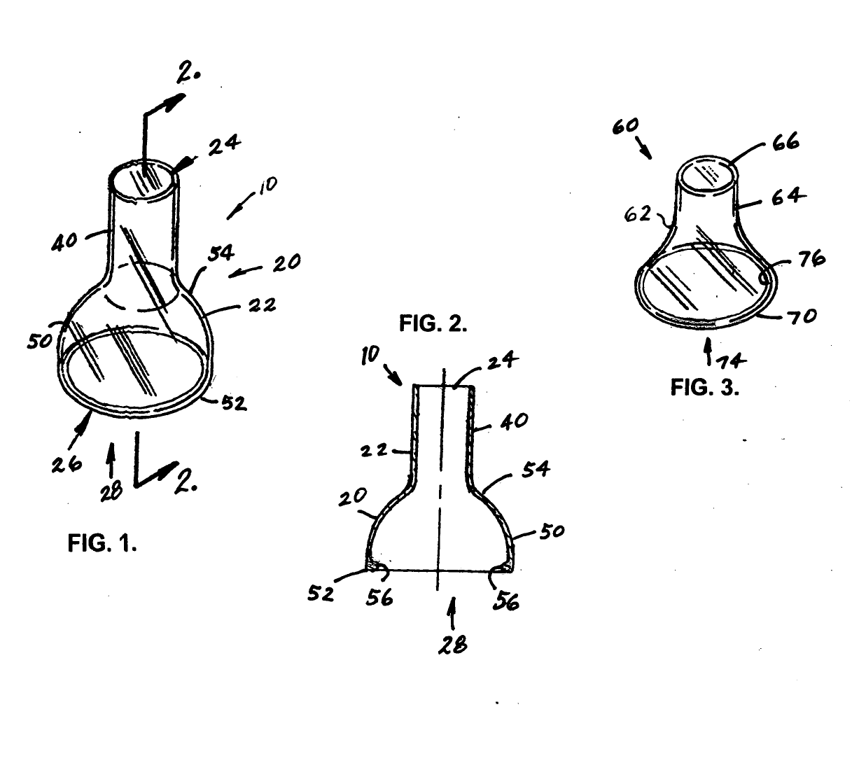

The reference numeral 10 generally refers to a nasal-dilating device in accordance with the present invention, as shown in FIGS. 1 and 2. The device 10 comprises a body unit 20 having a sidewall 22 with a circular cross-sectional shape, an open inner end 24, and an open outer end 26 such that air can freely pass lengthwise through the body unit 20, as indicated by the arrow designated by the numeral 28 in FIG. 2.

The body unit 20 has a unitary construction and is formed of flexible and resilient material, such as silicone rubber for example. Preferably, the body is constructed of material that is transparent or colored to blend with the skin tone of the nose of a user. The body unit 20 is dimensioned to be insertable into and frictionally retained in a constricted nostril of a user. The body unit 20 is laterally squeezable into an oval shape for purposes of inserting the body unit 20 into the constricted nostril and, upon being released after being inserted into the user's nostril, attempts to return to the circular cross-sectional shape thereof, thereby further opening the air passageway through the user's constricted nostril.

The body unit 20 may include a first portion 40 having a circular cross-sectional configuration and being substantially cylindrically shaped. The body unit 20 may also include a second portion 50 spaced in end to end relation with the first portion 40, the second portion 50 also having a circular cross-sectional configuration with a outer end 52. The second portion 50 extends radially outwardly from the first portion 40 and is shaped such that a shoulder 54 is formed adjacent to the first portion 40.

If desired, the body unit 20 may also include a peripheral ridge 56 extending radially inwardly from the outer end 52 of the second portion 50, as shown in FIG. 2. The peripheral ridge 56 serves at least two purposes: it enhances the ability of the body unit 20 to return to its circular cross-sectional shape after being released in a user's nostril as described herein, and it simplifies removal of the device 10 from the user's nostril.

The reference numeral 60 generally refers to a modified embodiment of the nasal-dilating device in accordance with the present invention, as shown in FIG. 3. The modified embodiment 60 comprises a body unit 62 having a sidewall 64 with a circular cross-sectional shape. The body unit 62 has an open inner end 66 and an open outer end 70 such that air can freely pass lengthwise through the body unit 62, as indicated by the arrow designated by the numeral 74 in FIG. 3. The body unit 62 flares continuously outwardly from the inner end 66 to the outer end 70. For some applications, the body unit 62 of the modified embodiment 60 may have a cross-sectional curvature that is approximately exponentially shaped.

If desired, the body unit 62 of the modified embodiment 60 may include a peripheral ridge 76 extending radially inwardly from the outer end 70, as shown in FIG. 3.

In an application of the present invention, a user places the device over the tip of the user's forefinger and secures the device with the thumb, such that the device assumes an oval shape. Then, while holding the device in the oval shape, the user slidably and gently inserts the inner end of the device into the constricted nostril and continues sliding the device into the constricted nostril until the device is fully inserted into his constricted nostril. As the device is being inserted into the constricted nostril, the device is gradually released. When the device is released from the oval shape, it attempts to return to the circular cross-sectional shape, thereby opening the air passageway through the user's constricted nostril. After insertion, the device may be rotated as necessary to assume a comfortable fit and to assure that the device is working properly. The device can be inserted or removed in public by disguising the action with a handkerchief or tissue.

For some applications, the device is inserted into the user's constricted nostril until the outer end thereof is spaced flush with an outer extremity of a user's constricted nostril. For other applications, it may be desirable to insert the device into the user's constricted nostril until the outer end thereof is spaced slightly inwardly from an outer extremity of a user's constricted nostril.

If desired, the device may be inserted such that the body unit rests on a ledge at the base of a user's columella/septum after being inserted into the user's nostril and released.

It is to be understood that if both of a user's nostrils are constricted, two of the nasal-dilating devices can be used, one in each of the two constricted nostrils.

It is also to be understood that while certain forms of the present invention have been illustrated and described herein, it is not to be limited to the specific forms or arrangement of parts as described and shown.

What is claimed and desired to be covered by Letters Patent is:

1. A nasal-dilating device for use with a constricted nostril, the device comprising a body unit having a sidewall, an open inner end and an open outer end such that air can freely pass through the device, the body unit having unitary construction and being formed of flexible and resilient material, wherein the body unit is dimensioned to be insertable into and frictionally retained in a constricted nostril, and wherein the body unit is laterally squeezable to an oval shape for purposes of inserting the body unit into the constricted nostril and, upon being released from the oval shape, attempts to return to the circular cross-sectional shape thereof, thereby opening the air passageway through the constricted nostril.

2. The nasal-dilating device as described in claim 1, wherein the body unit includes:
(a) a first portion having a circular cross-sectional configuration; and
(b) a second portion spaced in end to end relation with the first portion, the second portion having a circular cross-sectional configuration with a outer end and being configured to extend radially outwardly from the first portion.

3. The nasal-dilating device as described in claim 2, wherein the first portion is substantially cylindrically shaped.

4. The nasal-dilating device as described in claim 2, wherein the second portions is shaped such that a shoulder is formed therein, the shoulder being spaced adjacent to the first portion.

5. The nasal-dilating device as described in claim 1, wherein the body unit is dimensioned such that the outer end of the second portion is spaced slightly inwardly from an outer extremity of a user's nostril after being inserted into the user's nostril and released.

6. The nasal-dilating device as described in claim 1, wherein the body unit is dimensioned such that the outer end of the second portion is spaced flush with an outer extremity of a user's nostril after being inserted into the user's nostril and released.

7. The nasal-dilating device as described in claim 1, wherein the body unit includes a peripheral ridge extending radially inwardly from the outer end.

8. The nasal-dilating device as described in claim 1, wherein the body unit is dimensioned such that the body unit rests on a ledge at the base of a user's columella/septum after being inserted into the user's nostril and released.

9. The nasal-dilating device as described in claim 1, wherein the body unit flares continuously inwardly from the inner end to the outer end.

10. The nasal-dilating device as described in claim 9, wherein a cross-sectional curvature of the body unit is approximately exponentially shaped.

11. The nasal-dilating device as described in claim 9, wherein the body unit includes a peripheral ridge extending radially outwardly from the outer end thereof.

12. The nasal-dilating device as described in claim 1, wherein the body unit is constructed of silicone rubber.

13. The nasal-dilating device as described in claim 1, wherein the body unit is constructed of transparent material.

14. The nasal-dilating device as described in claim 1, wherein the body unit is colored to match the skin tone of the nose of a user.
